(19) United States
(12) Patent Application Publication
(10) Pub. No.: US 2026/0171255 A1
(43) Pub. Date: Jun. 18, 2026

(54) A MACHINE LEARNING PLATFORM FOR PREDICTING UROPATHOGENS AND THEIR RESISTANCE FOR PRESCRIBING SUITABLE URINARY INFECTION THERAPY

(71) Applicant: SRI SATHYA SAi INSTITUTE OF HIGHER LEARNING, Puttaparthi (IN)

(72) Inventors: Pradeep BULAGONDA ESWARAPPA, Puttaparthi (IN); Niranjana MAHALINGAM, Puttaparthi (IN); Balaram KHAMARI, Balangir dist (IN); Ratnakar PALAKODETI, Hyderabad (IN); Ramakumar KOMMAJOSYULA, Hyderabad (IN)

(21) Appl. No.: 18/996,500

(22) PCT Filed: Jul. 18, 2023
(86) PCT No.: PCT/IB2023/057299

§ 371 Date: Jan. 17, 2025

(30) Foreign Application Priority Data
Jul. 20, 2022 (IN) 202241041495

(51) Int. Cl. G16H 50/30; G16H 50/20; G16H 50/50; G16H 50/70

(52) U.S. Cl. CPC G16H 50/30; G16H 50/20; G16H 50/50; G16H 50/70

(57) ABSTRACT

The present invention provides a prediction model comprising a machine learning platform for differentiating high risk urine culture positive patients from those with negative culture. It also provides a platform to predict organism groups associated with UTI—based on patients' clinical history, comorbidities, and presenting symptoms.

### RELATED APPLICATION

This application is related to and claims priority from the Indian Provisional Application 202241041495 filed on 20 Jul. 2022 and is incorporated herein in its entirety.

### FIELD OF THE INVENTION

The present invention is related to a prediction model comprising a machine learning platform for differentiating high risk urine culture positive patients from those with negative culture. It also provides a platform to predict organism groups associated with UTI and their antibiotic susceptibility patterns—based on patients' clinical history, comorbidities, and presenting symptoms.

### BACKGROUND

Urinary Tract Infections (UTI) are widely prevalent globally leading to hospitalization, urosepsis and severe complications, especially in older people and pregnant women [1]. The clinical spectrum of UTIs range from asymptomatic bacteriuria, to symptomatic and recurrent UTIs, to sepsis associated with UTI that requires hospitalization [2][3]. However, delay in diagnosis is quite common in a large number of patients with asymptomatic bacteriuria or mild symptoms, resulting in further complications and prolonged/failed treatments [4]. Conversely, urine samples of a large number of suspected UTI patients are processed by hospitals every day which are avoidable [5]. Empirical treatment of such patients with unrequited antibiotics drives the selection and spread of antibiotic resistant uropathogens in the community. Non-treatment of asymptomatic bacteriuria is a vital opportunity for decreasing inappropriate antimicrobial use [5].

Antibiotics are the most effective and commonly prescribed drugs in the treatment of UTI: but, efficacy of antibiotics is dependent on how often they are being used and what fraction of these uropathogens have already acquired resistance against them. Enterobacteriaceae, a large family of Gram-negative bacteria that includes Escherichia coli and Klebsiella pneumoniae, is among the most prevalent causative organisms of UTIs [6][7][8]. β-lactam antibiotics have been commonly used as treatment options for UTIs associated with Enterobacteriaceae [9][10]. However, Extended Spectrum β-lactamase (ESBL) producing Enterobacteriaceae infections are of serious clinical concern as they can hydrolyse almost all the available i-lactam antibiotics [11]. Further, infections caused by ESBL producing Enterobacteriaceae have been reported to have higher morbidity and mortality.

If information regarding causative organisms and their antibiotic susceptibility patterns is available, effective alternate treatments can be prescribed. Unfortunately, procuring such information by processing patient samples in the microbiology labs may take between 24-48 hours, resulting in delayed or wrong treatment. To tackle this problem, a key step forward is early prediction of these incidences for timely prescription of appropriate antibiotics. Previous studies have investigated the prevalence, risk factors, and clinical features of typical and atypical UTIs (prediction of severity and mortality by APACHE scoring system [12], risk factors of urosepsis in older adults [3]). However, a definitive prediction tool that can differentiate patients with or without underlying UTI along with the organism class and their Antibiotic Susceptibility Test (AST) patterns purely based on clinical history and presenting symptoms is missing.

### SUMMARY OF THE INVENTION

In the current study, patient data based on an exhaustive list of features including presenting symptoms, comorbidities and clinical history was prospectively collected after informed consent from seven hospitals located in south India. This data was curated and used for the development of prediction model that can accurately predict UTI in suspected patients using only a set of clinical information. Further, machine learning models were developed which could predict whether a patient with a set of symptoms and comorbidities could be infected with an Enterobacteriaceae pathogen or not. Finally, if a patient is predicted to have an Enerobacteriaceae infection, an additional set of models were developed to predict the infecting Enterobacteriaceae to be a) ESBL-positive or negative among inpatients and outpatients separately and/or b) Nitrofurantoin resistant, and/or c) amikacin resistant, and/or d) Piperacillin_Tazobactum resistant and/or e) Cefoperzone_Sulbactum resistant, and/or f) Ciprofloxacin resistant, and/or g) Cefepime resistant, and/or h) Gentamicin resistant and/or i) Ceftriaxone resistant.

Upon successful implementation, this tool would save time, effort and resources, while also ensuring early prognosis and treatment of UTIs among patients who need it.

### BRIEF DESCRIPTION OF DRAWINGS

FIG. 1. Methodology followed for the development of prediction models.

FIG. 2. Distribution of urinary tract infection among males and females.

FIG. 3. Distribution of urinary tract infection across age groups.

FIG. 4. ROC curve of random forest model for the prediction of suspected urinary tract infections.

FIG. 5. Prevalence of UTIs caused by Enterobacteriaceae among male and female patients.

FIG. 6. Distribution of UTIs caused by Enterobacteriaceae across various age groups.

FIG. 7. ROC curve of random forest model for the prediction of Enterobacteriaceae among culture positive UTI patients.

FIG. 8. Prevalence of UTIs caused by ESBL-positive Enterobacteriaceae in males and females.

FIG. 9. Occurrence of UTIs caused by ESBL-positive Enterobacteriaceae versus ESBL-negative Enterobacteriaceae across age groups.

FIG. 10. ROC curve of inpatient random forest model for the prediction of ESBL producing Enterobacteriaceae.

FIG. 11. ROC curves of outpatient random forest model for the prediction of ESBL producing Enterobacteriaceae.

### DETAILED DESCRIPTION OF THE INVENTION

Claim 1: a Machine Learning Platform to Differentiate Patients with the Risk of Positive Urine Culture from Those Without—Based on their Clinical History, Comorbidities and Presenting Symptoms

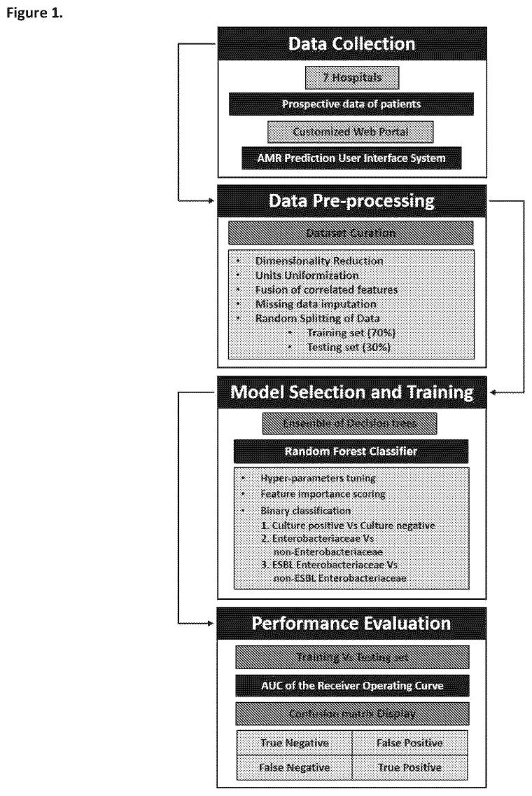

FIG. 1 provides the methodology followed for the development of prediction models.

### Methods: Data Collection

Prospective data of 4,136 patients (from 1 Apr. 2021 to 31 Mar. 2022) was collected from seven tertiary care hospitals located in South India viz. Sri Sathya Sai Institute of Higher Medical Sciences (Puttaparthi), NU Hospitals (Bengaluru), Sri Venkateswara Institute of Medical Sciences (Tirupati), Sri Ramachandra Medical College and Hospital (Chennai), Panimalar Medical College, Hospital and Research Institute (Chennai), Annapoorna Medical College and Hospital (Salem), and Vinayaka Mission's Kirupananda Variyar Medical College and Hospitals (Salem). A total of 170 features (variables), which included current symptoms, clinical history, age, marital status, number of children, etc. (Annexure 1) were collected from the patients along with their urine samples upon their consent to participate in the study. Urine samples were processed in the respective microbiology departments of each hospital to obtain culture results for all the patients. Data was entered into a secure custom-made web portal ‘AMR Prediction User Interface System’. (accessible at https://amrx.sssihl.edu.in/AMR/)

At a confidence interval of 95% (α=0.05), considering prevalence of UTIs to be 50% in patients who visit a hospital (P=0.5), and expecting at least 90% sensitivity and 90% specificity, the samples size requirement was calculated [13][14]. A minimum of 930 patients' data was required using this method. Alternately, for an expected AUC [Area Under the ROC (Receiver Operating Characteristic) Curve] of 0.95, the sample size was calculated to be a minimum of 1,584 patients [15].

### Data Pre-Processing

Patient records where urine culture reports were missing/unavailable were not included for the analysis. Dimensionality reduction was performed by merging two dependent features into a single one. Parameters containing values with multiple units were uniformized by performing necessary unit conversions. Absence of data for any symptom was assumed to be absence of the symptom and was evaluated accordingly. Highly correlated symptoms were combined and used for the calculation of a new feature that reflected these symptoms by a corresponding score. This resulted in 121 features being reduced to 73 (Supplementary Table 1). Each feature was converted into an appropriate category, integer or float depending on the nature of the data. There were some patients with asymptomatic urinary tract infections. Since asymptomatic UTIs are difficult to predict due to the absence of any clinical symptoms, such records were excluded from further analysis. For the remainder of the records, missing data for continuous values were imputed with their respective column medians. Thus, 3,848 patient records with 73 clinical parameters were finally utilized for building a machine learning model.

The entire data was split into two sets, one for training the model and another for testing the performance of the trained model. Data was randomly split into 70% training set and 30% testing set by invoking the train_test_split function from scikit-learn's model_selection module. Random_state was set at 1 to obtain the same split indefinitely.

### Prediction Modelling

Urine culture prediction is a binary classification problem (urine culture positive versus urine culture negative) for which Random Forest method was used. Random forest is an ensemble classifier in which the base concept is a decision tree. It is an ensemble of decision trees, where a series of decisions are made at each node depending on the selected parameters. Each record is classified into an output class (urine culture positive or urine culture negative) based on the decisions taken at every node. The samples and input parameters are bootstrapped to build uncorrelated trees in the forest. This allows each tree to be built independently using different sets of parameters and different sets of records. Random forest classifies every record into an output class based on the majority voting from all the decision trees of the forest.

Random forest classifier was imported from python's scikit-learn library that houses the ensemble module. Initially, all the 73 features were imported into the classifier with its default hyper-parameters to understand the performance of the classifier arbitrarily. The hyper-parameters were tuned to get optimum results. The hyper-parameter ‘criterion’ (default is ‘gini’) was set to ‘entropy’, ‘n-estimators’ (default is 100 trees) was set to 200, ‘max_features’ (default is ‘auto’) was set to ‘sqrt’, ‘max_depth’ (default is ‘none’) was set to 6, and ‘random_state’ was constantly set at 1 to obtain reproducible results for every run.

Random forest denotes the importance of each parameter with a feature importance score that is automatically calculated upon calling the ‘feature_importances_’ function. The features were sorted in the order of their feature importance scores and those having significant scores were selected as inputs to the model for further optimization. This process was repeated with different combinations of the features until the optimum set of features were obtained.

### Statistical Evaluation

AUC of the ROC curve was used as the performance metric to evaluate the performance of the model at every stage. Corresponding ROC curve was plotted using the “RocCurveDisplay” function from scikit-learn's metric module. From the same module, “ConfusionMatrixDisplay” function was used to get an account of the true positive, true negative, false positive and false negative count from a confusion matrix.

### Results

In total 4,079 urine culture reports were collected, of which 1,881 reports were urine culture positive whereas 2,198 reports were urine culture negative. This implies that about 53.9% of the patients did not have urinary infection although they were suspected to have an UTI. Early diagnosis of such patients saves the need for unnecessary laboratory investigations.

### Demographics

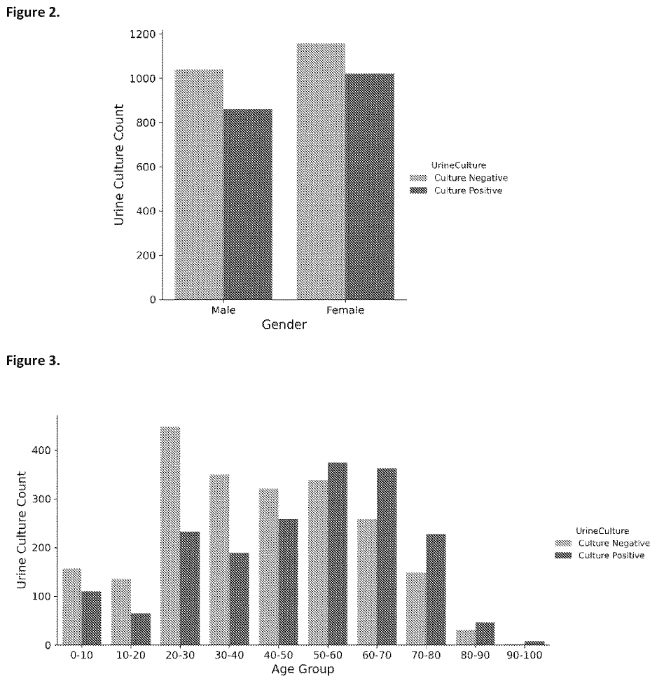

Of the 4,079 patients. 2,179 were females and 1,900 were males. 1,020 females were urine culture positive which constitutes about 46.8% of the female population whereas 861 males were urine culture positive which constitutes 45.3% of the males. This shows that both the genders have equally predisposed frequency for a urinary tract infection (FIG. 2).

It was observed that the ratio of UTI (56.7%) to non-UTI patients (43.3%) was much higher for the age group above 50 years indicating elderly people to be more susceptible to UTI. Meanwhile, the healthy population was constituted by the age group of 10-40 years, where the number of UTI cases (34.3%) were significantly lower than the number of non-UTI (culture negative) (65.7%) cases (FIG. 3).

### Prediction Modelling

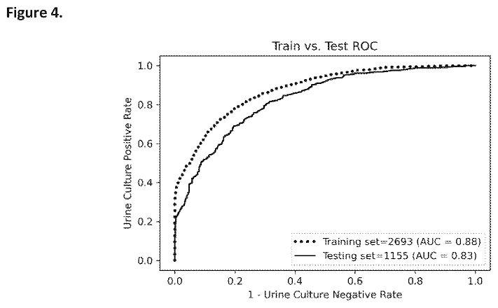

3,848 records were split into a training set of 2,693 and a testing set of 1,155 records. Both the training and testing sets had almost a balanced data of about 1:1 ratio with respect to urine culture positive and urine culture negative records. The training set utilized 30 out of the 73 features (Table 1-2) along with the tuned hyper-parameters to predict the output, i.e., urine culture positive or urine culture negative. The training set was imported into the random forest model with the optimized hyper-parameters and the model was fitted on this training data. The trained model was used to predict output for an unfamiliar test data. Based on the prediction probability, each record was assigned into an output class. The prediction probability was also used to compute the true positive rate and false positive rate over different thresholds for calculating the AUC score of the model using the ‘auc’ function from seikit-learn's ‘metries’ module. The AUC score of the train data is 0.88 and for the test data it is 0.83 (FIG. 4). Similarly, accuracy, precision and recall scores were computed using the predicted urine culture values and the actual urine culture values. The accuracy_score, precision_score and recall_score functions were used for this purpose. The performance metrics of the model with respect to the test data were given by an accuracy of 73.5%, precision of 0.79 and a recall of 0.63.

TABLE 1
List of Patient features used by the Random
Forest Model for UTI prediction

| S.L. No. | Patient features/symptoms |
|---|---|
| 1 | Age |
| 2 | Marital Status |
| 3 | Number of Children |
| 4 | Storage Symptoms |
| 5 | Voiding Symptoms |
| 6 | Dysuria |
| 7 | Foul Smelling Urine |
| 8 | Cloudy Urine |
| 9 | History of Fever and Chills |
| 10 | History of Generalized Weakness/Malaise |
| 11 | History of Nausea/Vomiting |
| 12 | History of Flank Pain |
| 13 | Length of stay in hospital |
| 14 | Surgical Status |
| 15 | First Time Hospitalisation - Duration of Stay |
| 16 | Pulse Rate |
| 17 | Systolic Blood Pressure |
| 18 | Diastolic Blood Pressure |
| 19 | Respiratory Rate |
| 20 | Temperature |
| 21 | Serum Creatinine |
| 22 | Haemoglobin |
| 23 | WBC Count |
| 24 | Neutrophil Count |
| 25 | Lymphocyte Count |
| 26 | Neutrophil to Lymphocyte Ratio |
| 27 | Pyuria |
| 28 | Bacteriuria |
| 29 | Inpatient (Yes or No) |
| 30 | Charlson's Comorbidity* |

*List provided in Table 2

TABLE 2
Patient features/symptoms used for calculation
of Charlson's Comorbidity index

| S.L. No. | Patient features/symptoms |
|---|---|
| 1 | Myocardial Infarction |
| 2 | Congestive Heart Failure |
| 3 | Peripheral Vascular Disease |
| 4 | Cerebrovascular Disease |
| 5 | Dementia |
| 6 | Chronic Pulmonary Disease |
| 7 | Connective Tissue Disease |
| 8 | Peptic Ulcer Disease |
| 9 | Mild Liver Disease |
| 10 | Diabetes without End Organ Damage |
| 11 | Hemiplegia |
| 12 | Moderate or Severe Renal Disease |
| 13 | Diabetes with End Organ Damage |
| 14 | Tumour without Metastases |
| 15 | Leukaemia |
| 16 | Lymphoma |
| 17 | Moderate or Severe Liver Disease |
| 18 | Metastatic Solid Tumour |
| 19 | AIDS |

### Conclusion

A prediction model was developed for the differentiation of probable UTI positive patients from UTI negative patients using random forest classifier with clinically acceptable sensitivity and specificity.

### Advantages of the Model

When compared with the currently practised laboratory methods, this machine learning tool is able to significantly reduce the investigation time, requirement for sophisticated instrumentation and skilled professionals. Further, this model would also reduce needless urine testing while also prompting urine test for high-risk patients.

Claim 2: A Machine Learning Platform that can Predict Organism Groups Associated with UTI—Based on Patients' Clinical History, Comorbidities, and Presenting Symptoms

### Methods: Data Pre-Processing

1.881 patients who were tested culture positive for a urinary tract infection (UTI) were filtered and their data was used in the building of a machine learning model for prediction of the infectious organism. 64 patient records which did not contain organism details were discarded leading to a final set of 1.817 records for analysis with 121 clinical parameters available against each record. Highly correlated symptoms were grouped into new features for the ease of calculation. This resulted in 121 features being reduced to 73 features. Each feature was converted into an appropriate category, integer or float data type depending upon the nature of the data of the specific parameter. Further, a new feature was created by categorizing infectious organisms as either belonging to Enterobacteriaceae family or non-Enterobacteriaceae family respectively. Outliers having aberrant clinical values were eliminated from further analysis resulting in 1,736 UTI patient records with 74 clinical parameters which were used for building the Enterobacteriaceae prediction machine learning model.

The organisms that were included as part of the Enterobacteriaceae group of pathogens were Escherichia coli, Klebsiella sp., Enterobacter sp., Citrobacter sp., Proteus sp., Morganella morganii, Serratia sp., and Providencia sp. All the other UTIs caused by any other organisms were grouped as non-Enterobacteriaceae. Since the data was imbalanced with respect to the infectious organism (Enterobacteriaceae count was 3.5 times higher than the non-Enterobacteriaceae count), RandomUnderSampler function from imblearn's under_sampling module was called to randomly under sample the majority class and balance the data. This balanced data was then randomly split into 70% training set and 30% testing set by invoking the train_test_split function from scikit-learn's model_selection module. Random_state was set at 1 to obtain the same under sampling and split indefinitely.

### Prediction Modelling

Univariate analysis of the features was performed using Pearson's correlation test. Features with continuous values were excluded from Pearson's correlation analysis. From stats module of ‘scipy’ library, the ‘pearsonr’ function was used to compute the Pearson's correlation coefficient of every feature with respect to the organism family. It also gave an insight into the statistical significance of each feature by providing a corresponding p-value. The features were sorted in the order of their p-values and those features having very low p-values were selected as inputs to the model for further optimization (Table 3). This process was repeated with different combinations of the features along with the continuous variables until an optimum set of features was arrived at. Ultimately, 17 out of the 74 features were found to give the most optimum result (Table 4). Enterobacteriaceae versus non-Enterobacteriaceae prediction is a binary classification problem for which Random Forest method was used. Random forest classifier was imported from python's scikit-learn library that houses the ensemble module. Initially, all the 74 features were imported into the classifier with its default hyper-parameters to understand the performance of the classifier arbitrarily. The hyper-parameters were tuned to get optimum results. The hyper-parameter ‘criterion’ (default is ‘gini’) was set to ‘entropy’, ‘n-estimators’ (default is 100 trees) was set to 110, ‘max_features’ (default is ‘auto’) was set to ‘log2’, ‘max_depth’ (default is ‘none’) was set to 8, and ‘random_state’ was set at 1 to obtain reproducible results for every run.

### Statistical Evaluation

AUC was used as the performance metric to evaluate the performance of the model at every stage. Corresponding ROC curve was plotted using the “RocCurveDisplay” function from scikit-learn's metric module. From the same module, “ConfusionMatrixDisplay” function was used to get an account of the true-positive, true-negative, false-positive and false-negative counts from the confusion matrix.

TABLE 3
Pearson's Correlation of the features for Enterobacteriaceae
prediction among culture positive patient records

| | Correlation | |
|---|---|---|
| Parameter | Coefficient | p-value |
| Voiding Symptoms | 0.209353 | 1.92 × 10⁻¹⁹ |
| HO Nausea Vomiting | 0.151501 | 8.53 × 10⁻¹¹ |
| HO Fever Chills | 0.145515 | 4.61 × 10⁻¹⁰ |
| Inpatient or Outpatient | −0.09257 | 7.75 × 10⁻⁰⁵ |
| HO Generalized Weakness/Malaise | 0.072976 | 0.001854 |
| Gender | 0.068899 | 0.003299 |
| Suprapubic Pain | 0.06479 | 0.005732 |
| Is Pregnant | 0.056649 | 0.015735 |
| Urologic Intervention in last 3 months | −0.05445 | 0.020277 |
| Pre-Surgery Urine Culture Organism Name | −0.04937 | 0.035336 |
| Surgical Status | −0.04804 | 0.040624 |
| Storage Symptoms | −0.04524 | 0.053837 |
| Foul Smelling Urine | 0.041752 | 0.075194 |
| Bacteriuria | −0.04102 | 0.080467 |
| HO Loss of Appetite | 0.040941 | 0.081041 |
| Haematuria | 0.040813 | 0.081991 |
| HO Catheterization | −0.03337 | 0.155061 |
| HO Sexual Exposure | 0.032328 | 0.168376 |
| Marital Status | 0.029887 | 0.202882 |
| Second Time Hospital Admission - Devices in-SITU | −0.02929 | 0.212082 |
| (Catheterized/Intubated) | | |
| HO Constipation | −0.02918 | 0.21384 |
| HO Tuberculosis | 0.025635 | 0.274757 |
| Gynaecological malignancy | −0.02544 | 0.278519 |
| Documentation of Infection within 1 Year | 0.024093 | 0.304694 |
| Endocrine Disorder | 0.019781 | 0.39939 |
| HO Previous UTI | 0.018308 | 0.435431 |
| Dysuria | 0.018174 | 0.438803 |
| Spinal Anomalies | −0.01798 | 0.443811 |
| Travel History within 2 weeks | 0.017775 | 0.448925 |
| Is he or she on prophylaxis | 0.017618 | 0.452941 |
| First Time Hospital Admission - Devices in-SITU | −0.0172 | 0.463679 |
| (Catheterized/Intubated) | | |
| Pre-Surgery Urine Culture Organism Group | 0.016159 | 0.491218 |
| Immunosuppressant Treatment within 1 Year | 0.015228 | 0.516539 |
| Hospital Type of Second Time Hospital Admission | −0.01445 | 0.53812 |
| Cloudy Urine | 0.012896 | 0.582759 |
| Pyuria | −0.01252 | 0.593881 |
| HO Testicular Pain or Mass | 0.012409 | 0.597074 |
| Reason for Surgery of Second Time Hospital Admission | −0.0107 | 0.648539 |
| Reason for Surgery of Third Time Hospital Admission | −0.0094 | 0.688847 |
| PriorUseOfSpecificAntibioticsWithin3 Months | −0.009 | 0.70139 |
| Anatomical Abnormality | −0.00857 | 0.714909 |
| Prophylactic Antibiotic | 0.007435 | 0.751453 |
| Devices in-situ | 0.007151 | 0.760658 |
| HO Flank Pain | −0.00519 | 0.824937 |
| Cystocele | 0.004528 | 0.847058 |
| Hospital Type of Third Time Hospital Admission | 0.003982 | 0.865299 |
| Hospital Type of First Time Hospital Admission | −0.00362 | 0.877393 |
| Recent Immunosuppressive Therapy/Chemotherapy | 0.001984 | 0.932662 |
| Reason for Surgery of First Time Hospital Admission | −0.00155 | 0.947483 |
| Third Time Hospital Admission - Devices in-SITU | 0.000762 | 0.97409 |
| (Catheterized/Intubated) | | |

### Results

In total 1,817 urine culture positive reports were collected, of which 1,405 reports were due to Enterobacteriaceae infections whereas 412 reports were associated with non-Enterobacteriaceae pathogens. This clearly exhibits that Enterobacteriaceae family is a common cause of a urinary tract infection (˜75%). This information holds tremendous potential during the prescription of antibiotics for the treatment of UTIs.

### Demographics

Of the 1,405 patients infected with an Enterobacteriaceae organism, 787 were females and 618 were males. On the other hand, out of the 412 patients infected with a non-Enterobacteriaceae organism, only 197 were females whereas 215 were males. This shows that females are more prone to an infection caused by an Enterobacteriaceae organism (FIG. 5).

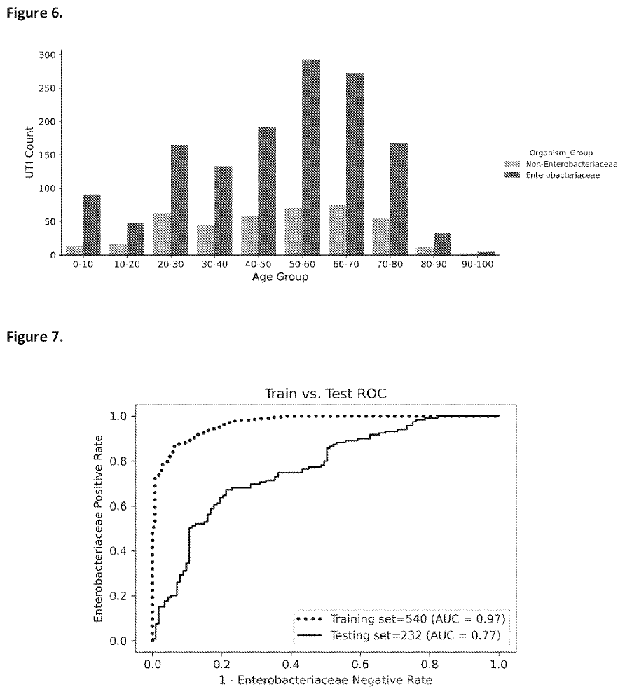

It was observed that the number of UTIs caused by Enterobacteriaceae was significantly higher than non-Enterobacteriaceae across all age groups. It was also observed that the number of infections were generally higher for the older age groups (50-70 years). The ratio of Enterobacteriaceae to non-Enterobacteriaceae in UTI patients was highest in the 50-70 age group and for children who were below 10 years of age (FIG. 6). These vulnerable groups should be tested for infections at the earliest or upon onset of symptoms.

### Prediction Modelling

1,736 records were under sampled with respect to Enterobacteriaceae count to obtain a balanced data set. This resulted in a total of 772 records of which 386 were Enterobacteriaceae and 386 were non-Enterobacteriaceae. These were then split into a training set of 540 records and a testing set of 232 records. The training set utilized 17 parameters (Table 4) to predict the output, Enterobacreriaceae or non-Enterobacteriaceae. The training set was imported into the random forest model with the optimized hyper-parameters and the model was fitted on this training data. The trained model was used to predict output for an unfamiliar test data. Based on the prediction probability, each record was assigned into an output class. The prediction probability was also used to compute the true-positive rate and false-positive rate over different thresholds for calculating the AUC score of the model using the ‘auc’ function from scikit-learn's metrics module. The AUC score of the train data is 0.97 and 0.77 for the test data (FIG. 7). Similarly, accuracy, precision and recall scores were computed using the predicted values and the actual values. The accuracy_score, precision_score and recall_score functions were used for this purpose. The performance metrics of the model with respect to the test data were given by an accuracy of 70.3%, precision of 0.72 and a recall of 0.69.

### Conclusion

Enterobacteriaceae prediction model was developed using Pearson's correlation analysis followed by random forest classifier for the differentiation of patients with Enterobacteriaceae infections from the patients with other UTIs (among confirmed UTI patients). Since majority of the UTIs are caused by Enterobacteriaceae, this prediction tool would significantly improve the treatment outcomes by supporting clinicians with scientific evidence and help in minimizing laboratory culture testing.

TABLE 4
List of Patient features used by the Random
Forest Model for organism prediction

| S.L. No. | Patient features/symptoms |
|---|---|
| 1 | Voiding Symptoms |
| 2 | Suprapubic Pain |
| 3 | Pulse Rate |
| 4 | History of Nausea/Vomiting |
| 5 | History of Fever/Chills |
| 6 | Inpatient or Outpatient |
| 7 | History of Generalized Weakness/Malaise |
| 8 | Pregnancy |
| 9 | Gender |
| 10 | Pre-urine culture organism ID |
| 11 | Urological intervention in last 3 months |
| 12 | Prior use of specific antibiotics within 3 months |
| 13 | Body Temperature |
| 14 | WBC Count |
| 15 | Diastolic Blood Pressure |
| 16 | Systolic Blood Pressure |
| 17 | Respiratory Rate |

### Claim 3: a Machine Learning Platform to Predict Antibiotic Resistance Patterns of Enterobacteriaceae—Based on Patients' Clinical History, Comorbidities, and Presenting Symptoms

### Methods: Data Pre-Processing

A total of 1,989 patients were UTI positive, of which 1,294 infections were caused by the Enterobacteriaceae. Data of these 1,294 patients was filtered to be used in the building of a machine learning model for the prediction of ESBL (Extended Spectrum β-lactamase) positive or ESBL negative organisms. 121 clinical parametere were used in the development of the prediction model. A new feature was created by categorizing each Enterohacteriaceae organism as either ESBL-positive or ESBL-negative (total 122 features). This served as the output variable for the prediction model. Highly correlated symptoms were grouped into new features. This resulted in 122 features being reduced to 73. The datasets were divided into multiple categories and analysed for efficient prediction. For example, the dataset was divided based on presence or absence of the following symptoms: a) hospitalization status, b) storage symptoms, c) voiding symptoms, d) haematuria, e) cloudy urine, f) devices in-SITU (catheterization or intubated), g) hospital type (private/public), h) bacteriuria, i) foul smelling urine, j) HO fever chills, k) dysuria, 1) HO nausea or vomiting, m) gender, n) anatomical abnormality, o) marital status, p) HO sexual exposure, q) reason for surgery, r) HO previous UTI, s) pyuria, t) history of catheterization. Analysis based on the above-mentioned divisions revealed that patient categories based on hospitalization status provided clinically meaningful results. The two distinct categories include ‘inpatient’ and ‘outpatient’. 67 features related to the outpatient dataset and an additional six features (totalling to 73 features) related to the inpatient dataset were used for ESBL prediction. The entire Enterobacteriaceae data was split into a training set for training the model and a testing set for testing the performance of the trained model. Since the data was imbalanced with respect to the ESBL positivity, it was balanced to obtain fair results. As the ESBL-positive count (763 nos.) was 1.4 times higher than the ESBL-negative count (531 nos.), “RandomUnderSampler” function from imblearn's under_sampling module was used to randomly under sample the majority class. This ensured that the ESBL-positive count matched the ESBL-negative count. Data was then randomly split into 70% training set and 30% testing set by invoking the train_test_split function from scikit-learn's model_selection module. Random_state was set at 1 to obtain the same under sampling and split indefinitely.

### Prediction Modelling

Random forest classifier was imported from python's scikit-learn library that houses the ensemble module. Two random forest models, one each for inpatient and outpatient were developed. Initially, all the 73 features for inpatient and 67 features for outpatient models were fed into the classifier with its default parameters to arbitrarily understand the performance of the model.

The hyper-parameters were tuned to get optimum results. The hyper-parameter ‘criterion’ (default is ‘gini’) was set to ‘entropy’, ‘n-estimators’ (default is 100 trees) was set to 200 for the inpatient model and 300 for the outpatient model, ‘max_features’ (default is ‘auto’) was set to ‘log2’, ‘max_depth’ (default is ‘none’) was set to 6, and ‘random_state’ was constantly set at 1 to obtain reproducible results for every run.
a) Inpatient model—Univariate analysis of the features was performed using Pearson's correlation test. Features with continuous values were excluded from Pearson's correlation analysis. From stats module of ‘scipy’ library, the ‘pearsonr’ function was used to compute the Pearson's correlation coefficient of every feature with respect to the ESBL status of the organism. It also gave an insight into the statistical significance of each feature by providing a corresponding p-value. The features were sorted in the order of their p-values and those features having very low p-values were selected as inputs to the model for further optimization. This process was repeated with different combinations of the features along with the continuous variables until an optimum set of features was arrived at (Table 5). Ultimately, 26 out of the 73 features along with the above tuned hyper-parameters were found to give the most optimum result for the “inpatient” model.
b) Outpatient model—Feature optimization of the outpatient model followed a different path. Random forest signifies the importance of each parameter with a feature importance score that is automatically calculated upon calling the feature_importances_function. The features were sorted in the order of their feature importance scores and those features having significant scores were selected as inputs to the model for further optimization. This process was repeated with different combinations of the features until an optimum set of features was obtained. Ultimately, 52 out of the 67 features along with the above tuned hyper-parameters were found to give the most optimum result for the “outpatient” model.
c) Prediction of individual antibiotic resistance

In addition to the prediction of ESBL and non-ESBL producing Enterobacteriaceae, further models were developed to predict whether a patient may harbour specific antibiotic resistant infections. The antibiotics with the maximum available patient data were selected for this project. Resistance predicted for the eight antibiotics were nitrofurantoin, amikacin, piperacillin-tazobactam, cefoperazone-sulbactam, ciprofloxacin, cefepime, gentamicin, and ceftriaxone. The basic methodology followed was similar to the previous predictions. A list of patients for whom a particular antibiotic data was available was segregated. The available data was divided into a training set and a testing set. The patient data of each antibiotic was also under-sampled to obtain a balanced data set. Both the under-sampled and total training set data were fed into the random forest model with optimized hyper-parameters and the model was fitted on this data.

### Statistical Evaluation

AUC was used as the performance metric to evaluate the performance of the model at every stage. Corresponding ROC curve was plotted using the “RocCurveDisplay” function from scikit-learn's metric module. From the same module, “ConfusionMatrixDisplay” function was used to get an account of the true-positive, true-negative, false-positive and false-negative counts from the confusion matrix.

TABLE 5
Pearson's Correlation of features related to
ESBL producing Enterobacteriaceae prediction

| | Correlation | |
|---|---|---|
| Parameter | Coefficient | p-value |
| Cloudy Urine | 0.174781 | 2.45 × 10⁻¹⁰ |
| Storage Symptoms | −0.16028 | 6.73 × 10⁻⁰⁹ |
| First Time Hospital Admission - Devices in-SITU | 0.12023 | 1.45 × 10⁻⁰⁵ |
| (Catheterized/Intubated) | | |
| Hospital Type of First Time Hospital Admission | 0.11828 | 1.99 × 10⁻⁰⁵ |
| HO Catheterization | 0.110512 | 6.78 × 10⁻⁰⁵ |
| Voiding Symptoms | −0.09995 | 0.000317 |
| Haematuria | 0.099724 | 0.000327 |
| Bacteriuria | 0.098293 | 0.000399 |
| Foul Smelling Urine | 0.094871 | 0.000633 |
| Urologic Intervention in last 3 Months | 0.092188 | 0.0009 |
| Dysuria | 0.090142 | 0.00117 |
| Second Time Hospital Admission - Devices in-SITU | 0.084039 | 0.002482 |
| (Catheterized/Intubated) | | |
| HO Fever Chills | 0.084034 | 0.002484 |
| Gender | −0.07984 | 0.004054 |
| HO Nausea/Vomiting | 0.078308 | 0.004825 |
| HO Previous UTI | −0.07648 | 0.005915 |
| Anatomical Abnormality | 0.074453 | 0.007376 |
| Marital Status | −0.07219 | 0.009385 |
| Reason for Surgery of First Time Hospital Admission | 0.072037 | 0.009536 |
| Hospital Type of Second Time Hospital Admission | 0.068177 | 0.014169 |
| Pyuria | 0.067201 | 0.015616 |
| Inpatient or Outpatient | 0.066671 | 0.016455 |
| HO Sexual Exposure | −0.05567 | 0.04526 |
| HO Flank Pain | −0.05516 | 0.047269 |
| Reason for Surgery of Second Time Hospital Admission | 0.053775 | 0.053121 |
| Documentation of Infection within 1 Year | 0.047099 | 0.090346 |
| Hospital Type of Third Time Hospital Admission | 0.046312 | 0.095871 |
| Prior Use of Specific Antibiotics within 3 Months | 0.042204 | 0.129173 |
| Suprapubic Pain | 0.039756 | 0.152924 |
| Reason for Surgery of Third Time Hospital Admission | 0.038532 | 0.165972 |
| Immunosuppressant Treatment within 1 Year | 0.034246 | 0.218298 |
| Recent Immunosuppressive Therapy/Chemotherapy | 0.034022 | 0.221321 |
| Is he or she on prophylaxis | −0.03317 | 0.233123 |
| Prophylactic Antibiotic | −0.02989 | 0.282705 |
| Surgical Status | −0.02906 | 0.296145 |
| Travel History within 2 Weeks | −0.02899 | 0.297381 |
| Pre-Surgery Urine Culture Organism Group | 0.025712 | 0.355398 |
| HO Generalized Weakness/Malaise | 0.02334 | 0.401533 |
| HO Loss of Appetite | 0.023318 | 0.401973 |
| Third Time Hospital Admission - Devices in-SITU | 0.018687 | 0.501818 |
| (Catheterized/Intubated) | | |
| Devices in-situ | 0.016471 | 0.553881 |
| Is Pregnant | 0.016305 | 0.557878 |
| HO Testicular Pain or Mass | 0.016083 | 0.563252 |
| Gynaecological Malignancy | −0.00755 | 0.786188 |
| Spinal Anomalies | 0.00717 | 0.796652 |
| Endocrine Disorder | 0.006606 | 0.812354 |
| HO Constipation | −0.00351 | 0.899618 |
| HO Tuberculosis | 0.0021 | 0.939828 |
| Cystocele | −0.00186 | 0.946767 |

### Results

In total 1,294 urine culture reports positive for Enterobacteriaceae were collected, of which 763 were positive for ESBL whereas 531 reports were negative for ESBL. This indicates that about 60% of the Enterobacteriaceae organisms that cause UTI are ESBL-positive. Antibiotic prescription for such resistant infections should be carried out diligently to have higher chances of recovery and avoid relapse.

### Demographics

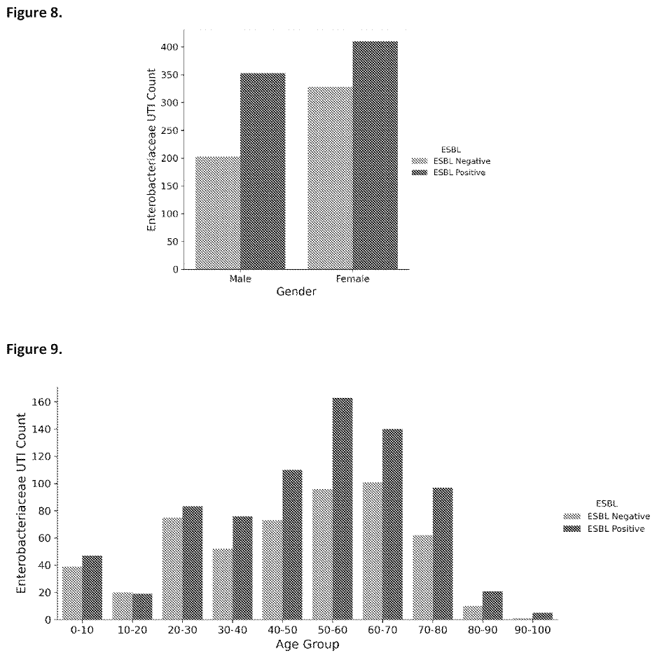

Of the 763 patients with ESBL-positive Enterobacteriaceae infections, 410 were females and 353 were males. On the other hand, of the 531 patients infected with non-ESBL Enterobacteriaceae, 328 were females and 203 were males. The proportion of ESBL-positive to ESBL-negative infections was found to be higher in males than in females. This indicates that an Enterobacteriaceae infection in males is more likely to be ESBL-positive (FIG. 8).

It was observed that ESBL-positive Enterobacteriaceae infections were significantly higher than non-ESBL infections in the 40-80 age group. Meanwhile, in the 0-30 age group both types of infections have almost equal chances of occurrence (FIG. 9). This strongly signifies that elderly people, who have an Enterobacteriaceae infection, are more likely to be antibiotic resistant.

### ESBL Prediction Models

The entire Enterobacteriaceae UTI data of 1,294 records was split into an “outpatient” category containing 754 records and an “inpatient” set containing 540 records.

### a) Inpatient Setting

The inpatient data was under-sampled with respect to ESBL-positive count to obtain a balanced data set. This resulted in a total of 406 records that were perfectly balanced. These were then split into a training set of 284 records and a test set of 122 records. The training set used 26 parameters (Table 6) to predict the output, i.e., ESBL-positive or ESBL-negative Enterobacteriaceae. The training set was fed into the random forest model with the optimized hyper-parameters and the model was fitted on this data.

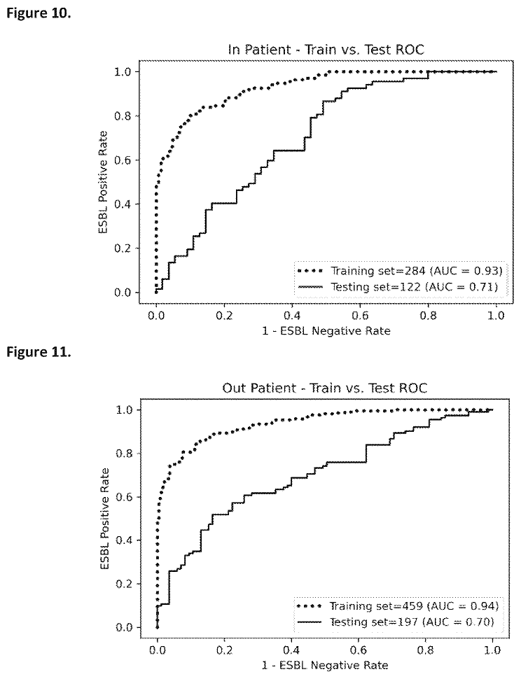

The trained model was used to predict output for an unfamiliar test data. Based on the prediction probability, each record was assigned into an output class. The AUC score for the train data was 0.93 and 0.71 for the test data (FIG. 10). The performance metrics of the model with respect to the test data were given by an accuracy of 61.5%, precision of 0.69 and a recall of 0.54.

TABLE 6
List of Patient features used by the Random Forest Model for prediction
of ESBL producing Enterobacteriaceae in “inpatient” group

| S.L No. | Patient features/symptoms |
|---|---|
| 1 | Cloudy Urine |
| 2 | Voiding Symptoms |
| 3 | Urological intervention in last 3 months |
| 4 | Anatomical Abnormality |
| 5 | Second Time Hospital Admission |
| 6 | Body Temperature |
| 7 | Storage Symptoms |
| 8 | First Time Devices In-situ (is Catheterized or Intubated) |
| 9 | First Time Hospital Admission |
| 10 | History of Catheterization |
| 11 | Bacteriuria |
| 12 | Haematuria |
| 13 | Foul Smelling Urine |
| 14 | History of Fever/Chills |
| 15 | Dysuria |
| 16 | History of Nausea/Vomiting |
| 17 | Second Time Devices In-situ (is Catheterized or Intubated) |
| 18 | Gender |
| 19 | Marital Status |
| 20 | History of Sexual Exposure |
| 21 | First Time Reason for Surgery |
| 22 | Pyuria |
| 23 | WBC Count |
| 24 | Inpatient or Outpatient |
| 25 | Second Time Duration of Catheterization |
| 26 | Haemoglobin |

### b) Outpatient Setting

The outpatient data was under-sampled with respect to ESBL-positive records count to obtain a balanced data set. This resulted in a total of 656 records that were perfectly balanced. These were then split into training set (459 nos.) and testing set (197 nos.). The training set utilized 52 parameters (Table 7) to predict the output (ESBL-positive or ESBL-negative Enterobacteriaceae). The training set was fed into the random forest model with the optimized hyper-parameters and the model was fitted on this training data.

The trained model was used to predict output for an unfamiliar test data. Based on the prediction probability, each record was assigned into an output class. The AUC score of the train data is 0.94 and 0.70 for the test data (FIG. 11). Similarly, accuracy, precision and recall scores were computed using the predicted and the actual values. The accuracy_score, precision_score and recall_score functions were aptly used for this purpose. The performance metrics of the model with respect to the test data were given by an accuracy of 65%, precision of 0.80 and a recall of 0.51.

TABLE 7
List of Patient features used by the Random Forest Model for prediction
of ESBL Enterobacteriaceae in an “outpatient” setting

| S.L No. | Patient features/symptoms |
|---|---|
| 1 | Age |
| 2 | Gender |
| 3 | Pregnancy |
| 4 | Marital Status |
| 5 | No of Children |
| 6 | Storage Symptoms |
| 7 | Voiding Symptoms |
| 8 | Dysuria |
| 9 | Suprapubic Pain |
| 10 | Foul Smelling Urine |
| 11 | Cloudy Urine |
| 12 | History of Fever/Chills |
| 13 | History of Generalized Weakness/Malaise |
| 14 | History of Nausea/Vomiting |
| 15 | History of Flank Pain |
| 16 | History of Loss of Appetite |
| 17 | History of Catheterization |
| 18 | Urological intervention in last 3 months |
| 19 | History of Previous UTI |
| 20 | Is he or she on prophylaxis |
| 21 | History of Tuberculosis |
| 22 | History of Sexual Exposure |
| 23 | Hospital Admission in 1 Year (Number of Times) |
| 24 | First Time Hospital Admission (Location) |
| 25 | First Time Hospital Admission (Duration) |
| 26 | First Time Devices In-situ (Is Catheterized or Intubated) |
| 27 | First Time Duration of Catheterization |
| 28 | Second Time Duration of Hospital Admission |
| 29 | Third Time Hospital Admission (Location and time of infection) |
| 30 | Third Time Duration of Hospital Admission |
| 31 | Prior Use of Specific Antibiotics within 3 Months |
| 32 | Immunosuppressant Treatment within 1 Year |
| 33 | Travel History within 2 Weeks |
| 34 | Endocrine Disorder |
| 35 | Pulse Rate |
| 36 | Systolic Blood Pressure |
| 37 | Diastolic Blood Pressure |
| 38 | Respiratory Rate |
| 39 | Body Temperature |
| 40 | Serum Creatinine |
| 41 | Haemoglobin |
| 42 | WBC Count |
| 43 | Neutrophil Count |
| 44 | Lymphocyte Count |
| 45 | Neutrophils-Lymphocytes Ratio |
| 46 | Pyuria |
| 47 | Bacteriuria |
| 48 | Haematuria |
| 49 | First Time Reason of Surgery |
| 50 | Second Time Reason of Surgery |
| 51 | Third Time Reason of Surgery |
| 52 | Charlson's Comorbidity Index* |

*List provided in Table 2

### c) Prediction of Individual Antibiotic Resistance

The trained model was used to predict output for an unfamiliar test data. Based on the prediction probability, each record was assigned into an output class. The best AUC score obtained was 0.66 for the under sampled test data of cefoperazone-sulbactam; whereas, high accuracy was observed for the test data of amikacin (80.2), cefoperazone-sulbactam (77.94), and piperacillin-tazobactam (75.62). Similarly, accuracy, true positive rate, and true negative rates were computed using the predicted and actual values (Table 8). The accuracy_score, precision_score and recall_score functions were used for this purpose.

TABLE 8
Development of evaluation of performance of prediction models developed
for individual antibiotics based on available patient data

| S. No. | Antibiotic | Train | Test | Under-sampling | Accuracy | TPR | TNR | AUC |
|---|---|---|---|---|---|---|---|---|
| 1. | Nitrofurantoin | 1827 | 784 | No | 70.41 | 85.32 | 19.66 | 60 |
| | | 817 | 351 | Yes | 54.99 | 76.61 | 34.44 | 59 |
| 2. | Amikacin | 1824 | 783 | No | 80.2 | 87 | 22.9 | 62 |
| | | 368 | 158 | Yes | 60.13 | 96.47 | 17.81 | 65 |
| 3. | Piperacillin-Tazobactam | 1779 | 763 | No | 75.62 | 92.04 | 13.75 | 64 |
| | | 749 | 321 | Yes | 55.76 | 88.34 | 22.15 | 61 |
| 4. | Cefoperazone-Sulbactam | 1745 | 748 | No | 77.94 | 89.5 | 22.48 | 64 |
| | | 593 | 255 | Yes | 60.39 | 86.76 | 30.25 | 66 |
| 5. | Ciprofloxacin | 1646 | 706 | No | 59.35 | 41.27 | 69.38 | 58 |
| | | 1124 | 482 | Yes | 52.49 | 87.1 | 15.81 | 55 |
| 6. | Cefepime | 1572 | 674 | No | 57.12 | 91.48 | 16.77 | 61 |
| | | 1391 | 597 | Yes | 50.25 | 84.9 | 15.72 | 56 |
| 7. | Gentamicin | 1541 | 661 | No | 68.84 | 85.16 | 21.3 | 58 |
| | | 767 | 329 | Yes | 51.37 | 86.59 | 16.36 | 59 |
| 8. | Ceftriaxone | 1372 | 589 | No | 46.86 | 79.82 | 26.04 | 58 |
| | | 1043 | 447 | Yes | 59.06 | 76 | 41.89 | 64 |

TPR, True-positive rate;
TNR, True-negative rate;
AUC, Area under the curve

### Conclusion

Two prediction models were developed for the differentiation of ESBL-positive and ESBL-negative Enterobacteriaceae infections. The first model was for inpatient settings, where univariate analysis followed by random forest classifier were used to select variables most correlated to ESBL-positive infections. In the second model for outpatient settings, the feature importance scores were directly calculated by random forest classifier. A third set of models help predict resistance against eight different antibiotics. These models hold tremendous potential in the prediction of antibiotic resistance among Enterobacteriaceae in UTI patients within a very short time and minimal effort. The conventional laboratory methods may take up to 48 hours for antibiotic susceptibility reporting, thus prompting clinicians to prescribe empirical therapy to minimize infections. Empirical therapy may or may not be successful while also increasing the rates of emergence of drug-resistant bacteria. Before prescribing a particular antibiotic, the clinicians can use this machine learning tool to assess the probability of encountering an antibiotic resistant infection and take a decision accordingly. Thus, these models can practically help clinicians move from empirical to evidence-based antibiotic therapy with minimal treatment-failures and reduction in the risk of further emergence of resistant bacteria.

SUPPLEMENTARY TABLE 1
List of clinical features used in the prediction models

| S.L | |
|---|---|
| No. | Patient features/symptoms |
| 1 | Age |
| 2 | Storage Symptoms |
| 3 | Hematuria |
| 4 | HO Generalized Weakness/Malaise |
| 5 | HO Loss of Appetite |
| 6 | HO Catheterization |
| 7 | Inpatient or Outpatient |
| 8 | Prophylactic Antibiotic |
| 9 | Is he or she on Prophylaxis |
| 10 | HO Tuberculosis |
| 11 | Hospital Type of First Time Hospital Admission (Private/Public) |
| 12 | First Time Hospital Admission - Devices in-SITU (Catheterized/ |
| | Intubated) |
| 13 | Duration of Catheterization of First Time Hospital Admission |
| 14 | Hospital Type of Second Time Hospital Admission |
| 15 | Second Time Hospital Admission - Devices in-SITU (Catheterized/ |
| | Intubated) |
| 16 | Duration of Catheterization of Second Time Hospital Admission |
| 17 | Hospital Type of Third Time Hospital Admission |
| 18 | Third Time Hospital Admission - Devices in-SITU (Catheterized/ |
| | Intubated) |
| 19 | Duration of Catheterization of Third Time Hospital Admission |
| 20 | Prior Use of Specific Antibiotics within 3 Months |
| 21 | Immunosuppressant Treatment within 1 Year |
| 22 | Recent Immunosuppressive Therapy/Chemotherapy |
| 23 | Pulse Rate |
| 24 | Serum Creatinine |
| 25 | Lymphocyte Count |
| 26 | Haematuria |
| 27 | Cystocele |
| 28 | Reason for Surgery of Second Time Hospital Admission |
| 29 | Charlson's Comorbidity |
| 30 | Gender |
| 31 | Voiding Symptoms |
| 32 | Foul Smelling Urine |
| 33 | HO Nausea/Vomiting |
| 34 | HO Constipation |
| 35 | Urologicintervention_in_last_3 months |
| 36 | Length of Stay in Hospital |
| 37 | Devices in-situ |
| 38 | Documentation of Infection within 1 Year |
| 39 | HO Sexual Exposure |
| 40 | Duration of First Time Hospital Admission |
| 41 | Duration of Second Time Hospital Admission |
| 42 | Duration of Third Time Hospital Admission |
| 43 | Travel History within 2 Weeks |
| 44 | Endocrine Disorder |
| 45 | Systolic Blood Pressure |
| 46 | Haemoglobin |
| 47 | Neutrophils-Lymphocytes Ratio |
| 48 | Urine Culture |
| 49 | Gynaecological Malignancy |
| 50 | Reason for Surgery of Third Time Hospital Admission |
| 51 | Anatomical Abnormality |
| 52 | Is Pregnant |
| 53 | Dysuria |
| 54 | Cloudy Urine |
| 55 | HO Flank Pain |
| 56 | HO Testicular Pain or Mass |
| 57 | Surgical Status |
| 58 | HO Previous UTI |
| 59 | Number of Times of Hospital Admission in 1 Year |
| 60 | Number of Children |
| 61 | Temperature |
| 62 | Respiratory Rate |
| 63 | Neutrophil Count |
| 64 | Bacteriuria |
| 65 | Spinal Anomalies |
| 66 | Marital Status |
| 67 | Suprapubic Pain |
| 68 | HO Fever Chills |
| 69 | Diastolic Blood Pressure |
| 70 | White Blood Cells Count |
| 71 | Pyuria |
| 72 | Patient Unique ID |
| 73 | Reason for Surgery of First Time Hospital Admission |

### Annexure 1: Summary of the AMR Patient Questionnaire

All responses contained in the questionnaire are strictly confidential and are part of patient's medical record. The questionnaire includes information related to the
Personal data that includes Name, Age, Gender (with pregnancy/menopausal status), Marital Status, Mobile Number, No of Children, and address. (This information is de-identified)
Presenting Complaints including any Storage Symptoms, voiding symptoms, dysuria, suprapubic pain, hematuria, foul smell in the urine, history of catheterization, urological intervention in the last three months.
If the patient is an inpatient, information related to admission to ward/ICU, dates of admission, length of hospital stay, devices in situ, surgeries, pre-op and post-op urine status and antibiotics used.
Past Infection data contains history of previous infection within three months, no. of times, any prophylactic treatments given, history of infection within 1 year, history of Tuberculosis, history of sexual exposure.
Hospital Admission history includes admissions to hospital within 1 year, and the details thereof (location of hospital, reason of admission, surgeries performed, duration of hospital stay, devices in situ, catheterization status)
Drug history includes names of antibiotics, immunosuppressants used previously (within 3 months and within a year)
Travel History that inquires if the patient travelled out of his hometown in the past two weeks.
Information related to patients comorbidities included Myocardial infection, Congestive heart failure, Peripheral vascular disease, Cerebrovascular disease, Dementia, Chronic pulmonary disease, Connective tissue disease, Peptic ulcer disease, Mild liver disease, Diabetes without end-organ damage, Hemiplegia, Moderate or severe renal disease, Diabetes with end-organ damage, Tumor without metastases, Leukemia, Lymphoma, Moderate or severe liver disease, Metastatic solid tumor, AIDS, Recent immunosuppressive therapy /chemotherapy, Endocrine disorder (Hypothyroid etc.), Any Others.
Clinical Parameters including pulse rate, BP, respiration, body temperature and other clinical investigation that include Serum creatinine, Hemoglobin. WBC count, Neutrophil count, Lymphocyte count, Neutrophil/lymphocyte ratio, CRP, Pyuria, Bacteriuria, Hematuria, Urine culture report (if any), Blood culture report (if any).
Anatomical Abnormalities on Imaging that include urological (Urolithiasis, Tumors of the urinary tract, Ureteric strictures, UPJO, urethral stricture, Neurogenic bladder, Renal cysts. Posterior urethral valve, Vesicoureteral reflux, Bladder Diverticula, Nephrocalcinosis, Prostatic hypertrophy, Diverticula, Pelvicalyceal obstruction, Congenital abnormalities, Indwelling urethral catheter, Intermittent catheterization. Ureteric stent, Nephrostomy tube. Urological procedures, Ileal conduit, Medullary sponge kidney, Renal failure, Renal transplant) and non-urological (spine anomalies, cystocele, and gynecological malignancy)

Each patient questionnaire was signed by the patient after written informed consent and reviewed by the clinician before submission.

### REFERENCES

[1]T. L. Griebling, “Re: Charactenstics of Febrile Urinary Tract Infections in Older Male Adults,” J. Urol., vol. 204, no. 3, p. 595, 2020, doi: 10.1097/JU.0000000000001163.01.
[2]L. Mody and M. Juthani-Mehta, “Urinary tract infections in older women: A clinical review,” JAMA—J. Am. Med. Assoc., vol. 311. no. 8. pp. 844-854.2014. doi: 10.1001/jama.2014.303.
[3]B. C. Peach. G. J. Garvan. C. S. Garvan, and J. P. Cimiotti. “Risk Factors for Urosepsis in Older Adults,” Gerontol. Geriatr. Med., vol. 2. p. 233372141663898, 2016. doi: 10.1177/2333721416638980.
[4]J. Komagamine, T. Yabuki, D. Noritomi, and T. Okabe, “Prevalence of and factors associated with atypical presentation in bacteremic urinary tract infection,” Sci. Rep., vol. 12, no. 1. pp. 1-6, 2022, doi: 10.1038/s41598-022-09222-9.
[5]L. E. Nicolle et al., “Clinical practice guideline for the management of asymptomatic bacteriuria: 2019 update by the Infectious Diseases Society of America,” Clin. Infect. Dis., vol. 68. no. 10, pp. E83-E75, 2019. doi: 10.1093/cid/ciy1121.
[6] World Health Organization, Prioritization of Pathogens to Guide Discovery. Research and Development of New Antibiotics for Drug-resistant Bacterial Infections, including Tuberculosis. Geneva. Switzerland: World Health Organization. 2017.
[7]D. van Duin and D. L. Paterson. “Multidrug-Resistant Bacteria in the Community: Trends and Lessons Learned,” Infect. Dis. Clin. North Am., vol. 30, no. 2, pp. 377-390, 2016. doi: https://doi.org/10.1016/j.idc.2016.02.004.
[8]S. Mohd Sazlly Lim, P. L. Wong. H. Sulaiman, N. Atiya, R. Hisham Shunmugam. and S. M. Liew, “Clinical prediction models for ESBL-Enterobacteriaceae colonization or infection: a systematic review,” J. Hosp. Infect., vol. 102. no. 1. pp. 8-16, 2019. doi: https://doi.org/10.1016/j.jhin.2019.01.012.
[9]P. D L. and B. R. A., “Extended-Spectrum β-Lactamases: a Clinical Update,” Clin. Microbiol. Rev., vol. 18. no. 4. pp. 657-686. Oct. 2005, doi: 10.1128/CMR.18.4.657-686.2005.
[10]D. S. Teklu. A. A. Negeri, M. H. Legese. T. L. Bedada. H. K. Woldemariam, and K. D. Tullu. “Extended-spectrum beta-lactamase production and multi-drug resistance among Enterobacteriaceae isolated in Addis Ababa, Ethiopia,” Antimicrob. Resist. Infect. Control, vol. R. no. 1. p. 39, 2019, doi: 10.1186/s13756-019-0488-4.
[11]J. D. D. Pitout and K. B. Laupland, “Extended-spectrum β-lactamase-producing Enterobacteriaceae: an emerging public-health concern.” Lancet Infect. Dis., vol. 8, no. 3, pp. 159-166, 2008. doi: https://doi.org/10.1016/S1473-3099(08)70041-0.
[12]S. VijayGanapathy, V. S. Karthikeyan, J. Sreenivas, A. Mallya, and R. Keshavamurthy, “Validation of APACHE II scoring system at 24 hours after admission as a prognostic tool in urosepsis: A prospective observational study,” Investig. Clin. Urol., vol. 58, no. 6, pp. 453-459, 2017. doi: 10.4111/icu.2017.58.6.453.
[13]K. Hajian-Tilaki, “Sample size estimation in diagnostic test studies of biomedical informatics,” J. Biomed. Inform., vol. 48. pp. 193-204, 2014. doi: 10.1016/j jbi.2014.02.013.
[14]A. Negida, N. K. Fahim, and Y. Negida, “Sample Size Calculation Guide—Part 4: How to Calculate the Sample Size for a Diagnostic Test Accuracy Study based on Sensitivity. Specificity, and the Area Under the ROC Curve.,” Adv. J. Emerg. Med., vol. 3. no. 3, p. e33, 2019, doi: 10.22114/ajem.v0i0.158.
[15]G. Lu, “Sample Size Formulas For Estimating Areas Under the Receiver,” 2021.

We claim:

1. A prediction model comprising a machine learning platform to differentiate patients with the risk of positive urine culture versus those without the risk, wherein the said method is based on a combination of attributes derived from the patients.

2. The prediction model as claimed in claim 1, wherein the said attributes are clinical history, comorbidities and presenting symptoms.

3. The prediction model as claimed in claim 2 wherein the said comorbidities are patient's features as listed in Table 2.

4. The prediction model as claimed in claim 2 wherein the said presenting symptoms are patient's features as listed in Table 1.

5. A prediction model comprising a machine learning platform to predict organism groups associated with urinary tract infections (UTI) based on a combination of attributes derived from the patients.

6. The prediction model comprising a machine learning platform to predict organism groups as claimed in claim 5 wherein the said attributes are clinical history, comorbidities and presenting symptoms.

7. The prediction model as claimed in claim 5, wherein the said organism group is Enterobacteriaceae group of pathogens.

8. The prediction model as claimed in claim 7, wherein the said Enterobacteriaceae group of pathogens is selected from Escherichia coli, Klebsiella sp., Enterobacter sp., Citrobacter sp., Proteus sp., Morganella morganii, Serratia sp., and Providencia sp.

9. The prediction model as claimed in claim 7, wherein the features for Enterobacteriaceae group of pathogens are selected from the culture positive patient records as listed in Table 3.

10. A prediction model comprising a machine learning platform to predict antibiotic resistance patterns of Enterobacteriaceae based on a combination of attributes derived from the patients.

11. The prediction model comprising a machine learning platform to predict antibiotic resistance patterns of Enterobacteriaceae as claimed in claim 10 wherein the said attributes are clinical history, comorbidities and presenting symptoms.

12. A prediction model comprising a machine learning platform as claimed in claims 1, 5 and 10, consisting the steps of:
a. Data collection from customized web portal;
b. Data pre-processing and dataset curation;
c. Model selection and training using random forest classifier; and
d. Performance evaluation.
